(19) United States
(12) Patent Application Publication
(10) Pub. No.: US 2006/0241339 A1
(43) Pub. Date: Oct. 26, 2006

(54) Method and apparatus for an adjustable implantable continence device

(76) Inventors: Timothy C. Cook, Wayzata, MN (US); John H. Burton, Minnetonka, MN (US)

Correspondence Address: Schwegman, Lundberg, woessner & Kluth, P.A., P.O. Box 2938, Minneapolis, MN, 55402, US

(21) Appl. No.: 11/361,016

(22) Filed: Feb. 23, 2006

Related U.S. Application Data
(60) Provisional application No. 60/655,356, filed on Feb. 23, 2005.

(51) Int. Cl. A61F 2/04

(52) U.S. Cl. 600/31

(57) ABSTRACT

The present subject matter includes an implantable device and method for restoring continence and controlling coaptivity of a body lumen. The device includes a fill port block which has one or more self-sealing septums. The fill-port block is connected to one or more elongate conduits and ultimately to one or more expandable elements for communicating fluid. The one or more expandable elements are implanted and adjusted from a remote location using the fill-port block. Methods for implantation, use, and construction are also provided.

### CLAIM OF PRIORITY

This application claims the benefit of U.S. Provisional Patent Application Ser. No. 60/655,356 filed Feb. 23, 2005, entitled: “Method and Apparatus for an Adjustable Implantable Continence Device,” which is incorporated by reference in its entirety.

### TECHNICAL FIELD

This document relates generally to method and apparatus for implantable devices, and in particular to method and apparatus for an adjustable implantable continence device.

### BACKGROUND

Various implantable devices and methods have been attempted to restore continence to patients. One type of incontinence a patient suffers from is fecal incontinence. One such attempt is the use of an active adjustment cuff for constricting the anal canal in an effort to regain continence. Active adjustment cuffs are adjusted by the patient prior to and after each voiding process. Improperly used, such cuffs can cause tissue ischemia and tissue erosion, which can lead to other complications and health problems.

What is needed in the art is a system which restores the natural function of the human anatomy in the voiding process. Such a system should require no intervention by the patient, yet be adjustable by a physician over time with minimal invasiveness to avoid tissue ischemia and tissue erosion.

### SUMMARY

The above-mentioned problems and others not expressly discussed herein are addressed by the present subject matter. The present subject matter includes in varying embodiments apparatus for controllably coapting a body lumen including: an expandable element; a fill port block including a self-sealing septum; and a conduit providing fluid communication between the fill port block and the expandable element, wherein the expandable element is adapted to become toroidal in shape when at least partially filled and wherein the apparatus is adapted for implantation. The apparatus include variations wherein the expandable element is adapted to expand to a predetermined shape. Examples constructed using silicone elastomer, polyurethane elastomer, among other things, are provided. Various embodiments include a needle stop in the septum. It is understood that additional expandable elements can be added to the apparatus, and that such elements can be connected in parallel to the septum or connected in series to the septum. Multiple septums may be employed. Different shapes are contemplated. Options for different shaped expandable elements include cylindrical or curved, or asymmetrical, among other things. In various embodiments, the conduits can vary between 5 and 20 centimeters. Variations are also provided for expandable elements having curved shapes with the foregoing variations, except that the different shaped expandable elements include toroidal, cylindrical and asymmetrical shapes.

Also provided are novel apparatus for filling a plurality of septums of a fill port, including: a hypodermic needle body; and needle means for communicating fluid with the plurality of septums of the fill port. Different needles and methods are provided.

Also provided are methods of implantation, use and methods for manufacturing the components, including: connecting a plurality of adjustable elements to a plurality of conduits; and connecting at least one conduit of the plurality of conduits to a self-sealing septum to form a closed system which is adapted for adjusting volume of the adjustable elements based on fluids exchanged between the system and an external source.

This Summary is an overview of some of the teachings of the present application and not intended to be an exclusive or exhaustive treatment of the present subject matter. Further details about the present subject matter are found in the detailed description and appended claims. The scope of the present invention is defined by the appended claims and their legal equivalents.

### BRIEF DESCRIPTION OF THE DRAWINGS

FIG. 1 illustrates a cross section side view of a continence device, according to one embodiment of the present subject matter;

FIG. 2 illustrates a partial cross-section of an expandable element of a continence device demonstrating an element approximating a toroidal shape, according to one embodiment of the present subject matter;

FIG. 3 illustrates a partial cross-section view of an expandable element of a continence device demonstrating an element approximating a cylindrical shape, according to one embodiment of the present subject matter;

FIG. 4 illustrates a partial cross-section view of an expandable element of a continence device demonstrating an element approximating a curved shape, according to one embodiment of the present subject matter;

FIG. 5 illustrates a partial cross-section view of an expandable element of a continence device demonstrating an element having an asymmetrical shape, according to one embodiment of the present subject matter;

FIG. 6A illustrates a schematic of an implantable device, according to one embodiment of the present subject matter;

FIG. 6B illustrates a schematic of an implantable device, according to one embodiment of the present subject matter;

FIG. 7 illustrates a method of using an apparatus to coapt a body lumen, according to one embodiment of the present subject matter;

FIG. 8A illustrates a top view of a continence device according to one embodiment of the present subject matter;

FIG. 8B illustrates a cross section at line 8B of FIG. 8A, according to one embodiment of the present subject matter;

FIG. 9 illustrates a perspective view of a delivery tool used for implantation of a continence device, according to one embodiment of the present subject matter;

FIG. 10 illustrates a perspective view of a tool used for creating a passage in tissue of a patient, according to one embodiment of the present subject matter;

FIG. 11 illustrates a front view of a tool used for dilating tissue during implantation of an implantable device, according to one embodiment of the present subject matter;

FIG. 12A illustrates a front view cross section of an apparatus for adjusting a continence device, according to one embodiment of the present subject matter;

FIG. 12B illustrates a side view cross section of an apparatus for adjusting a continence device, according to one embodiment of the present subject matter;

FIG. 12C illustrates a side view cross section of an apparatus for adjusting a continence device, the cross section taken at line 12C in FIG. 12A;

FIG. 13A illustrates a front view of an apparatus for adjusting a continence device, according to one embodiment of the present subject matter;

FIG. 13B illustrates a side view of an apparatus for adjusting a continence device, according to one embodiment of the present subject matter;

FIG. 13C illustrates a system for adjusting a continence device, according to one embodiment of the present subject matter;

FIGS. 14A illustrates a front view of an element for implantation in a patient, according to one embodiment of the present subject matter;

FIGS. 14B illustrates an end view of an element for implantation in a patient, according to one embodiment of the present subject matter;

FIG. 15A illustrates an implanted device and a funnel shaped anal canal, according to one embodiment of the present subject matter;

FIG. 15B illustrates one method for fecal continence, according to one embodiment of the present subject matter;

FIG. 16A illustrates an implanted device and a coapted anal canal, according to one embodiment of the present subject matter;

FIG. 16B illustrates one method for fecal continence, according to one embodiment of the present subject matter;

FIG. 17 illustrates a cross section of an implanted device and a coapted anal canal, according to one embodiment of the present subject matter;

FIG. 18 illustrates a schematic of an implanted fecal continence device, according to one embodiment of the present subject matter;

FIG. 19 illustrates a method for using anal manometry in implantation of a fecal continence device, according to one embodiment of the present subject matter;

FIG. 20A illustrates a cross section of a patient, according to one embodiment of the present subject matter; and

FIG. 20B illustrates a method for using a caudal incision for implantation of a fecal continence device, according to one embodiment of the present subject matter.

### DETAILED DESCRIPTION

The following detailed description of the present subject matter refers to subject matter in the accompanying drawings which show, by way of illustration, specific aspects and embodiments in which the present subject matter may be practiced. These embodiments are described in sufficient detail to enable those skilled in the art to practice the present subject matter. References to “an”, “one”, or “various” embodiments in this disclosure are not necessarily to the same embodiment, and such references contemplate more than one embodiment. The following detailed description is demonstrative and not to be taken in a limiting sense. The scope of the present subject matter is defined by the appended claims, along with the full scope of legal equivalents to which such claims are entitled.

The present subject matter includes method and apparatus for a continence device. In various embodiments, the subject matter includes a fecal continence device. Childbirth, aging, surgery, trauma, and other patient experiences can affect anatomical function. The present method and apparatus offer a solution that provides continence in a variety of patients by controllable and adjustable coaptation of a subject's anal canal. The present system is designed to reduce tissue ischemia and resulting tissue erosion and return normal function to the patient. Some embodiments of the present system are also designed to reduce unwanted device migration and offer novel implantation methods and apparatus.

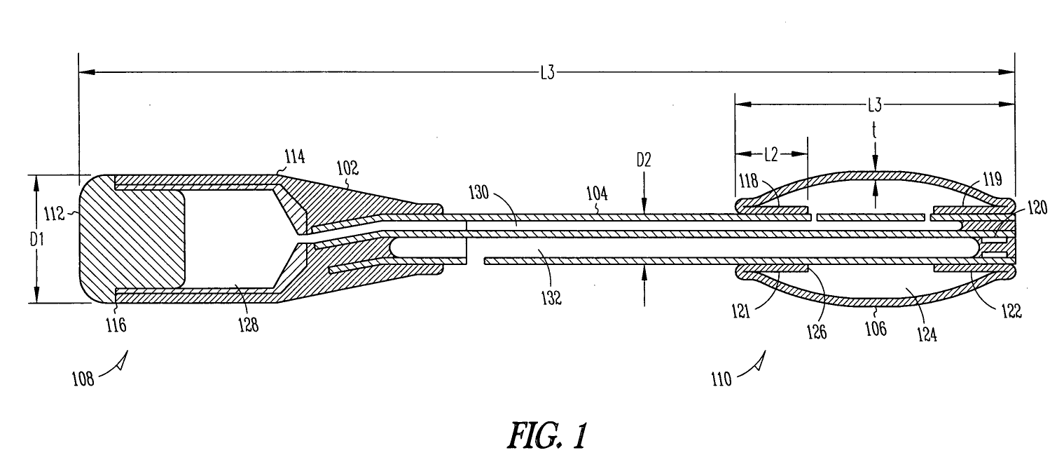

FIG. 1 illustrates a cross section side view of an implantable device for coaptation of a body lumen, according to one embodiment of the present subject matter. In various applications, one or more of the illustrated devices coapt the anal canal. The example illustrated is elongate, and includes a near portion 108 and a far portion 110. It should be noted that the example pictured is only one example of a device for anal coaptation, and other shapes and configurations are within the scope of the present subject matter.

In various embodiments, the implantable device includes a fill-port block 102 at the near portion 108. The fill-port block 102 is adapted to receive a needle of an external device, such as a syringe, to add or subtract flowable material and thereby change the volume 124 defined by the expandable element 106. In various embodiments, the fill-port block 102 includes a self-sealing septum 112 which seals after a needle is removed from it. In one embodiment the self-sealing septum 112 includes silicone elastomer. In one embodiment, the self-sealing septum 112 includes polyurethane elastomer. Other shapes, materials, configurations and combinations may be used which are within the scope of the present subject matter.

The self-sealing septum 112 conforms to and is fixed to the fill-port block 102. One embodiment fixes the septum with an adhesive 116 such as MED2-4213 adhesive from NUSIL SILICONE TECHNOLGY of Carpinteria, Calif. Other types of self-sealing septa and adhesives may be used without departing from the present subject matter.

In varying embodiments, the fill-port block 102 includes an insert 114. In some embodiments, the insert 114 is rigid to serve as a needle stop. In one embodiment, the fill-port block 102 includes a titanium insert, also termed a port jacket, which is enveloped by silicone from NUSIL SILICONE TECHNOLOGY of Carpinteria, Calif. One embodiment is enveloped with NUSIL MED-4840 LSR type silicone, but the present subject matter is not so limited.

The fill-port block 102 in conjunction with the self-sealing septum 112 and insert 114 defines a chamber 128. In one example, the fill-port block 102 is substantially cylinder shaped at the near portion 108, and has a diameter D1 ranging from about 0.15 inches to 0.40 inches. In one embodiment, the apparatus has a diameter D1 ranging from about 0.220 inches to about 0.230 inches. These sizes are for illustration and not intended to be exhaustive or exclusive. In some embodiments one or more flat sides are used to provide better grasp of the fill port block 102. Other shapes and diameters may be used without departing from the present subject matter.

Various embodiments include an elongate conduit 104. The elongate conduit is flexible, in various embodiments. In various examples, elongate conduit 104 includes a first lumen 130 in fluid communication with chamber 128. Optionally, in various embodiments, elongate conduit 104 includes a second lumen 132 which is adapted to receive a push-wire. In one embodiment elongate conduit 104 includes a silicone elastomer material. In one embodiment elongate conduit 104 includes a polyurethane elastomer material. Other types of conduits having different materials, shapes, and features are possible within the scope of the present subject matter.

Fill-port block 102, combined with an elongate conduit 104, has an overall approximate length of L1. The length may vary and is adapted to provide adequate distance between the situs of the expandable element 106 and a typically subcutaneous situs of the self-sealing septum 112. Thus, various lengths are contemplated. Lengths of a plurality of devices may vary in length to accommodate varying distances for various element sites and various septum sites. In various embodiments, the length ranges from about 5.0 centimeters to about 20.0 centimeters. In various embodiments, L1 includes, but is not limited to, any of the following approximate lengths: about 5, 6, 7, 8, 9, 10, 11, 12, 13, 14, 15, 16, 17, 18, 19, or 20 centimeters. However, it is understood that other lengths may be used that are too numerous to mention expressly.

The present subject matter includes embodiments having a cylindrical elongate conduit 104 with a diameter D2. Diameters can vary provided that the inner lumen 130 of the conduit 104 can effectively communicate flowable material between the self-sealing septum 112 and the volume 124. The diameter is generally kept to a minimum possible to allow for flexibility of the device and minimal invasiveness to a subject. In varying embodiments, diameter D2 ranges from about 0.092 inches to about 0.098 inches. However, it is understood that other diameters can be used in varying embodiments which are too numerous to mention herein expressly. The shape of the lumen 130 is cylindrical in one embodiment. Other shapes may be employed without departing from the scope of the present subject matter. In some of these embodiments, lumen 130 ranges from about 0.017 inches to about 0.019 inches in diameter. Second lumen 132 is additionally cylindrical in various embodiments. In some of these embodiments, second lumen 132 has a diameter from about 0.039 inches to about 0.041 inches. It is understood that other shapes and dimensions may be employed without departing from the present subject matter and that such shapes and dimensions are too numerous to all be expressly stated herein.

Various embodiments include an expandable element 106 at or near the far portion 110. In varying embodiments, the element membrane 106 is silicone elastomer. In various embodiments, other materials are used, such as urethane. Some embodiments include a substantially uniform thickness t. The thickness t can vary provided that the expandable element 106 can achieve its desired inflated and uninflated shapes. For example, some embodiments employ thickness t ranges from about 0.007 inches to about 0.015 inches. Other embodiments do not have a substantially uniform thickness t, and the resulting structure can have a variety of thicknesses distributed over the element 106. It is also noted that element membrane thicknesses can vary with inflation, so the numbers given herein are only an approximation and are not intended to be exclusive or exhaustive.

One method of manufacturing expandable element 106 includes repeated dipping of a mandrel into silicone which is not cured, which forms an expandable element 106 on the mandrel. The expandable element 106 is then cured and removed from the mandrel and connected to the other parts of the device. In various embodiments, the mandrel is dipped multiple times to achieve varying thicknesses of the expandable element 106. Some portions of the mandrel may be dipped in uncured silicone more than others, resulting in thickness t which is not uniform. This manufacturing method provides for customization using fewer manufacturing resources, including less complex tooling changes.

In various embodiments, the expandable element 106 is attached to an elongate conduit 104 at first joint 118 and second joint 119. In some embodiments, the expandable element 106 and elongate conduit 104 are silicone, and joints 118, 119 include silicone adhesive 121, 122. One adhesive used is MED2-4213 adhesive from NUSIL SILICONE TECHNOLGY of Carpinteria, Calif., but other adhesives are within the scope of the present subject matter.

In various embodiments, the expandable element 106 defines a volume 124. Addition of flowable material to the volume 124 to inflate the expandable element 106 subjects joints 118, 119 to various stresses which may result in an unwanted separation of the joints between the expandable element 106 and the elongate conduit 104. In one example embodiment these stresses are reduced by directing edge 126 inward, toward the volume 124, as shown in FIG. 1. In one embodiment, joint 119 extends along the elongate conduit 104 an approximate distance of L2. In some embodiments, L2 ranges from about 0.116 inches to about 0.140 inches. Additionally, in some embodiments, expandable element 106 extends along elongate conduit 104 a distance of L3. In varying embodiments, dimension L3 ranges from about 0.587 inches to about 0.627 inches. Other distances are possible without departing from the scope of the present subject matter. Thus, those presented herein are intended to provide a range which is demonstrative and not intended to be exclusive or exhaustive.

In some embodiments, the far portion 110 of the implantable device includes a radioopaque marker 120 used during fluoroscopy. While not absolutely necessary, such markings can assist in placement of the device or monitoring its operation later while in vivo.

Various types of flowable materials can be used to adjust the expandable element 106. One type of flowable material that can be used is an isotonic solution. One application includes and isotonic solution having a combination of sterile water and contrast material. Some embodiments include flowable materials which are radioopaque or include radioopaque elements. The flowable material is added or withdrawn with an external source. In one embodiment, a syringe having a hypodermic needle is used to adjust the expandable element 106 and thus, coaptation. In embodiments having one or more septa implanted subcutaneously, the needle can be used to adjust one or more expandable elements 106 without requiring surgery to access the one or more septa. Other flowable materials and external sources may be used without departing from the scope of the present application.

Expandable element 106 provides a compact form for easy insertion during implantation. Once in position, the expandable element 106 is adjustable. In various embodiments, the element 106 can be adjusted to any number of volumes by injecting flowable material. In various embodiments, the element 106 is adapted for variation between an initial shape and a predetermined use shape. The use shape in various embodiments is adjustable to provide controllable coaptation of a body lumen. Variable sizes and postoperative adjustability of the device provide on-going treatment customization to maintain a more natural lumen coaptation and to address anatomical changes.

In various embodiments, expandable element 106 is elastic. In various embodiments, expandable element 106 is substantially elastic, and expands like a balloon. In various embodiments, expandable element 106 is substantially inelastic and expands to a predetermined shape. This is accomplished through selective application and choice of various polymeric compounds, in various embodiments, to produce an expandable element 106 which expands to a substantially predetermined shape. One material includes, but is not limited to, high modulus polyurethane (PET). Embodiments using materials which range between elastic and inelastic are within the scope of the present subject matter, and these examples are not exhaustive or exclusive of the present subject matter. The enumerated configurations are provided for demonstration, and are not exhaustive or exclusive of configurations within the scope of the present subject matter.

In varying applications of the present subject matter the expandable element 106 abuts the anal canal of a subject, and the fill-port block 102 is positioned subcutaneously for easy adjustment during implantation and postoperatively without additional surgery. Some positionings of the block, include but are not limited to, the labia in females and the scrotum in males.

Adjustment of the device can occur at various times. For example, an initial adjustment is made during implantation to set a preliminary coaptation of the body lumen. Such settings may be limited in case the patient experiences swelling from edema. Additional adjustments can be performed after surgery to establish more normal operation of the body lumen, and hence continence. In some cases, adjustment can be made approximately four to approximately six weeks after implantation to compensate for anatomical changes related to edema. This time can also be used to allow for encapsulation of the apparatus to prevent migration. Adjustment can occur throughout the patient's life to compensate for future anatomical changes. One benefit of the device is that such adjustments are performed using a syringe and a small gauge needle, without surgery. One example of a small gauge needle includes a 23 gauge needle.

FIGS. 2-5 illustrate varying examples of expandable element 106. The different shapes demonstrate only some options provided to a physician for implantation. Various different shapes may be used in combination to tailor the tissue bulking effect, and thus the coaptation of a lumen. Some expandable elements are specially designed prior to implantation to inflate to predetermined use shapes. By selecting specific materials and material thicknesses comprising the expandable element, various shapes are accomplished. Pre-molded shapes designed to inflate to a predetermined shape are used in some embodiments. Expandable elements with structures influencing expansion, such as bands, are used in additional embodiments. Expandable elements with varying thicknesses, causing diverse rates of expansion during inflation are used in some embodiments. Various embodiments include materials which are elastic, and are more readily deformable by the local anatomy than more rigid designs. These examples serve as illustrations of the scope of the present subject matter, and are not exhaustive or exclusive of the present subject matter.

FIG. 2 illustrates a partial cross-section of an expandable element of a continence device demonstrating a element approximating a toroidal shape, according to one embodiment of the present subject matter. The expandable element 206 is attached to an elongate conduit 204, in various embodiments. In various embodiments, dimension L21 is less than dimension L22.

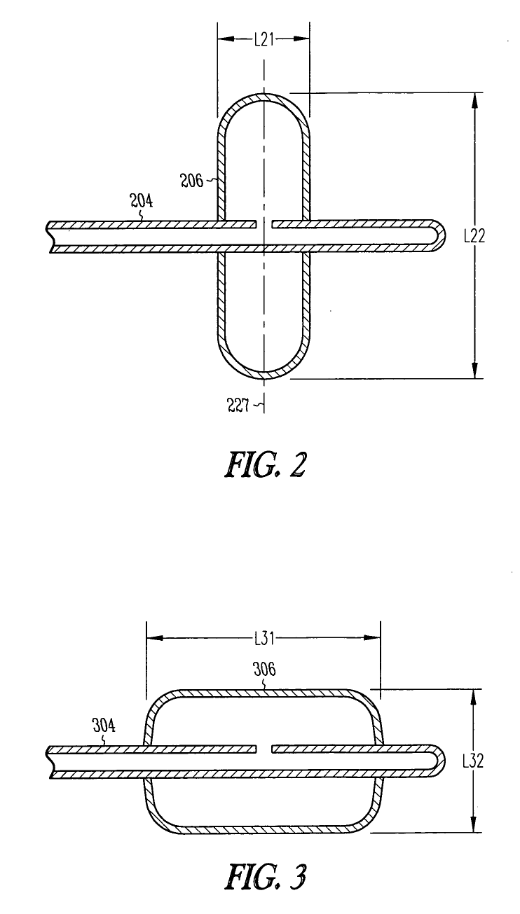

FIG. 3 illustrates a partial cross-section view of an expandable element 306 of a continence device demonstrating a element approximating a cylindrical shape, according to one embodiment of the present subject matter. The expandable element 306 is attached to an elongate conduit 304, in various embodiments. In various embodiments, dimension L31 is larger than dimension L32. Elongate expandable element 306 aids a user in placement of the expandable element 306. For example, when positioning the device, a physician can achieve coaptation in an improved number of implantation configurations, as the expandable element 306 can be placed along a side of the targeted body lumen at various positions along the length L31.

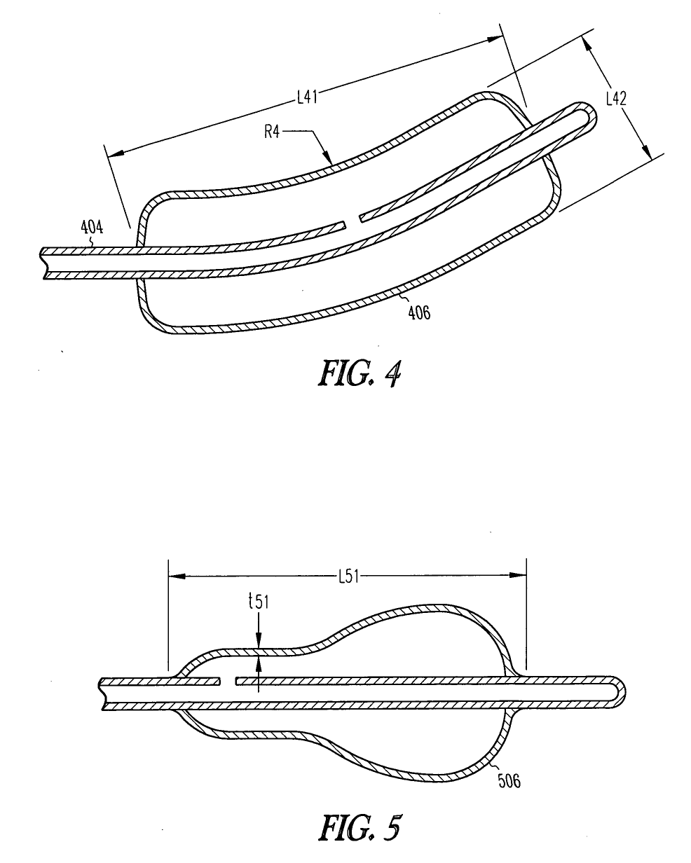

FIG. 4 illustrates a partial cross-section view of an expandable element 406 having an asymmetrically curved shape, according to one embodiment of the present subject matter. Various embodiments include symmetrically curved shaped embodiments, straight embodiments, and other embodiments not listed here. The expandable element 406 is attached to an elongate conduit 404, in various embodiments. Various examples include a constant radius dimension R4. Some embodiments include a variable radius dimension R4, as measured along the length L41. In various embodiments, length L41 is greater than thickness L42, but the present subject matter is not limited to this configuration. Curved expandable elements allow a physician to implant a device in a patient with varied anatomy. Additionally, a curved expandable element 406 can extend to anatomical regions which are not reachable by a physician. For example, when implanting through a straight and narrow passage, the curved expandable element 406 can extend outside the envelope defined by the straight and narrow passage.

FIG. 5 illustrates a partial cross-section view of an expandable element 506 of a continence device demonstrating a element having an asymmetrical shape, according to one embodiment of the present subject matter. In various embodiments, the expandable element 506 has a varying thickness t51, resulting in an irregular shape. It is understood that expandable element 506 may also be formed of a less elastic material to expand to a predetermined shape. Irregular shapes are useful for adapting the coaptation system to varied anatomy.

Various embodiments of expandable element 506 have circular cross-sections. Some examples include bands to achieve multiple diameters along the length L51 of the expandable element. In some embodiments, multiple diameters are achieved using various expandable element 506 thicknesses. Other structures achieve multiple diameters, and these enumerated structures should not be interpreted as exhaustive or exclusive of the present subject matter.

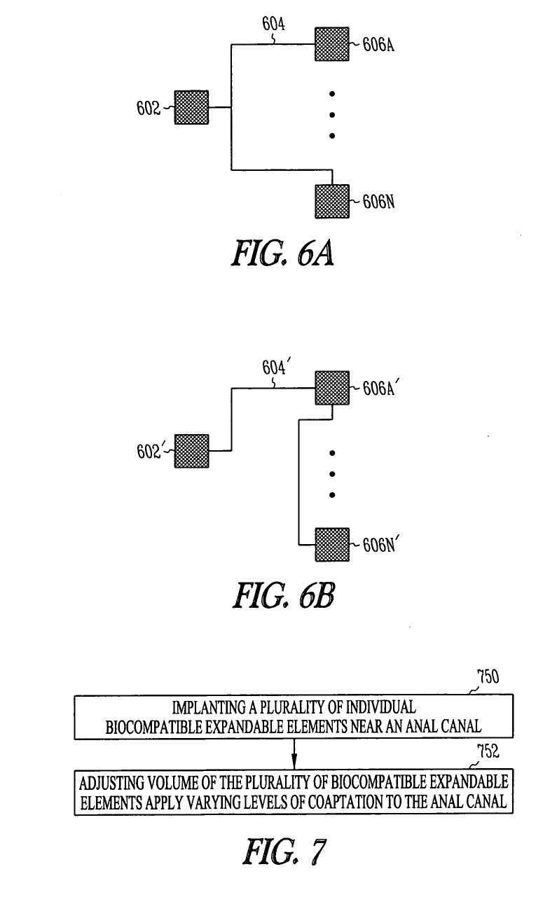

FIG. 6A illustrates a functional schematic of an implantable device, according to one embodiment of the present subject matter. Various device structures are available within the scope of the present subject matter to achieve the illustrated functionality. The system includes a fill-port 602, including one or more self-sealing septums defining one or more chambers. The system also includes a conduit 604 for connecting the fill-port to at least one expandable element 606A. Various embodiments have two or more expandable elements 606A, . . . , 606N. In various embodiments, the chambers of the fill-port area 602 are in fluid communication with volumes defined by the one or more expandable elements 606A, . . . , 606N. In various embodiments, the one or more expandable elements 606A, . . . , 606N are implanted to coapt a body lumen. In one example, one expandable element 606A is placed near a body lumen. In additional embodiments, multiple expandable elements 606A, . . . , 606N are placed near a single body lumen. In some embodiments, two or more expandable elements 606A, . . . , 606N are placed near two or more body lumens. It is understood that some embodiments may incorporate a number of different shapes of expandable elements. In some embodiments, the shapes are the same.

The schematic illustrates a fill-port block 602 used in conjunction with a plurality of expandable elements 606A, . . . , 606N. In various embodiments, the number and shape of the expandable elements are selected based on a particular patient's anatomy. The distance separating the fill-port block 602 and an expandable element, such as expandable element 606A, is selected based on a particular patient's anatomy and the sites of implantation of the expandable element and the septum. In various embodiments, the distance between a first expandable element 606A and a second expandable element is selected based on a particular patient's anatomy and the placement of the expandable elements and septum.

In one example, a system for fecal continence including three expandable elements is implanted around the anal canal. Each expandable element may be independently adjustable to restore function as normal and thereby restore continence. Additional embodiments adapted to patient anatomy are within the scope of the present subject matter.

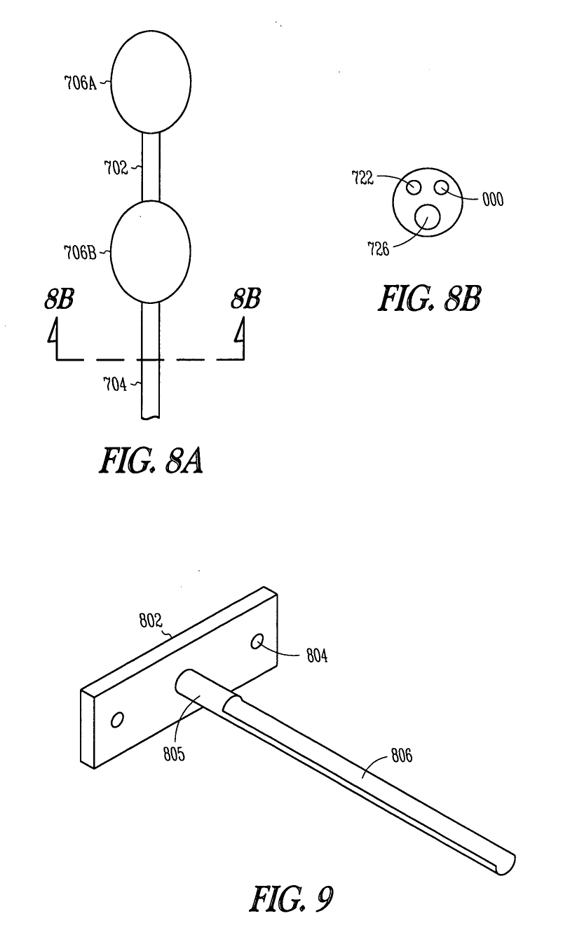

It is noted that FIG. 6A is intended to show only some of the possible configurations of the present subject matter. Other embodiments incorporating single elements, such as multiple devices as shown in FIG. 1 can be positioned separately. Embodiments having multiple elements in a linear configuration, such as the design of FIGS. 8A and 8B, are also possible. Other combinations are possible without departing from the scope of the present subject matter. For example, FIG. 6B illustrates a functional schematic of an implantable device, according to another embodiment of the present subject matter. The schematic includes a fill-port 602′, including one or more self-sealing septums defining one or more chambers. The system also includes a conduit 604′ for connecting the fill-port to at least one expandable element 606A′. Various embodiments have two or more expandable elements 606A′, . . . , 606N′. Various embodiments have expandable elements of the same shape. Various embodiments have expandable elements of different shapes.

FIG. 7 illustrates a method of using an apparatus to coapt a body lumen, according to the present subject matter. In various embodiments, the method includes a first step 750 of implanting a plurality of expandable elements near an anal canal. Additionally, in various embodiments, the method includes a second step 752 of adjusting volume of the plurality of expandable elements to apply varying levels of coaptation to the anal canal. In varying embodiments, a physician may adjust one or more expandable elements to achieve varied levels of coaptation to restore proper voiding function and perhaps accommodate patient preference.

FIG. 8A illustrates a top view of a continence device according to one embodiment of the present subject matter. To illustrate how the functional schematic illustrated in FIG. 6 applies to various structures, the present FIG. 8 uses numbers which match that schematic. But it should be noted that the scope of example schematic FIG. 6 is not so limited.

The illustrated example device includes two expandable elements 606A, 606B, attached to an elongate conduit 604. These elements are unfilled during implantation, but are shown in their at least partially expanded form. The device also includes a secondary conduit 702. In various applications, a passage is created in the tissue of a patient, and the expandable elements 606A and 606B are positioned near a body lumen by insertion through the passage. The shape of the example demonstrates multiple elements in a linear configuration a less invasive implantation in a single passage. The number of elements and their sizes may vary without departing from the scope of the present subject matter.

FIG. 8B illustrates a cross section at line 8B of FIG. 8A, according to one embodiment of the present subject matter. The example cross section approximates one shape the device assumes during implantation. This implantation configuration is relatively long and narrow, and adapted for insertion through a long and narrow passage. During implantation, a tissue incision is made to permit the perimeter of the cross-section 8B to enter into tissue. The two expandable elements, in the example, are deflated for passage.

The cross section includes a first lumen 722 through which a flowable material passes for inflation and deflation of an expandable element. A second lumen 724 communicates a flowable material for inflation and deflation of an expandable element. Also visible is a lumen 726 for use with a push-wire or a guidewire to assist in implantation of the device.

FIG. 8B illustrates a cross section at line 8B of FIG. 8A, according to one embodiment of the present subject matter. The example cross section approximates one shape the device may embody. Other shapes are possible without departing from the scope of the present subject matter. The cross section includes a first lumen 722 through which a flowable material passes for inflation and deflation of an expandable element. Also visible is a lumen 726 for use with a push-wire or a guidewire to assist in implantation of the device. In single fill lumen embodiments, first lumen 722 can expand both expandable element 706A and 706B. Various embodiments include a second lumen 724 to allow for independent adjustment of expandable element 706A and expandable element 706B. Thus, various embodiments feature different adjustability of different expandable elements.

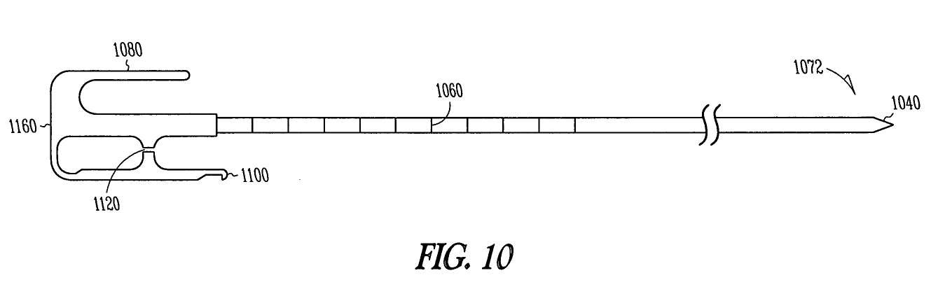

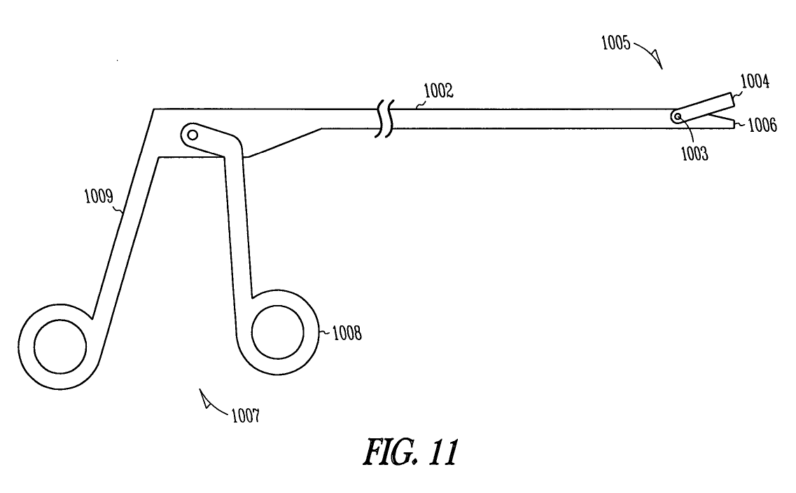

Various delivery tools may be employed, including but not limited to those provided by U.S. patent application Ser. No. 11/226,519, filed Sep. 14, 2005, which is hereby incorporated by reference in its entirety. FIG. 9 illustrates a perspective view of a delivery tool, according to one embodiment of the present subject matter. The first section 805 is constructed to fully encircle objects passing through it, such as a stylet. The second section 806 is adapted for partially encircling any objects passing through it. Various embodiments of the delivery tool include a face 802 which is used for manipulation of the delivery tool in some methods. Some embodiments include a catch 804 for locking additional apparatus to the face 802, such as a stylet (one example is pictured in FIG. 10) or a tissue expanding device (one example is pictured in FIG. 11).

In various embodiments, the passage defined by section 806 and section 805 is useful for inserting an implantable device in a patient. When inserted in a patient the delivery tool defines a space through which an implantable device may pass into a use position. Various embodiments use a guidewire or push-wire to locate the implantable device into a use position.

Some examples of the delivery tool include additional forms and methods for device location. Physical features included on the delivery tool, in various embodiments, indicate device location during surgery. For example, following insertion, a user can palpate the patient's tissue nearby the delivery tool and associated locating features to understand delivery tool orientation. Such features include protrusions and other physical shapes. Additionally included in some examples are delivery tool shapes which project as distinct during ultrasound procedures. Of further use during ultrasound procedures are various marker materials disposed on the delivery tool, the materials having compositions which project as distinct.

FIG. 10 illustrates a top view of a stylet, according to one embodiment of the present subject matter. In varying embodiments, the stylet includes an alignment feature 1080 and a locking barb 1100, in addition to a flex-stop 1120. In varying examples, the alignment feature 1080 is useful for mating to a delivery tool. In additional embodiments, the alignment feature 1080 is useful as a register to stop axial movement of the stylet along the lengthwise center axis of the stylet.

In varying designs, the stylet lock 1160 includes features which partially enable locking of the stylet with other equipment. In various embodiments the locking barb 1100 elastically flexes to lock to a delivery tool. The flex-stop 1120 is useful to prevent plastic deformation of the stylet.

The stylet, in varying designs, is useful for puncturing tissue. To this end, various embodiments of the stylet include splines 1040 useful for tissue tunneling. Additionally, the stylet includes varying tip designs at the far portion 1072. For example, in varying embodiments, the stylet includes a sharp tip such as a pin-point. In additional embodiments, the stylet includes a blunt tip, which is useful for creating a passageway in tissue with reduced instances of unwanted tissue puncture. The stylet, in various embodiments, includes markings 1060 useful for measuring the distance of stylet insertion.

FIG. 11 illustrates a front view of a tool used for dilating tissue to accommodate implantation of an implantable device, according to one embodiment of the present subject matter. In some embodiments, the tool includes a graspable handle located at a near portion 1007. The graspable handle, in varying embodiments, includes a static handle member 1009 and a pivot handle member 1008. Additionally, the tool includes jaws at a far portion 1005, and an elongate shaft 1002 disposed between the graspable handle and the jaws. In varying embodiments, moving the pivot handle 1008 towards handle member 1009 opens the jaws 1004, 1006 to dilate tissue. Moving pivot handle 1008 away from handle member 1009 closes jaws 1004, 1006. In varying designs, this is accomplished through linkage mechanisms. In varying embodiments, the jaws include a static jaw 1006 which is statically fixed to the elongate shaft 1002 and a pivot jaw 1004 which is rotatably mounted to the elongate shaft 1002. In varying embodiments, the pivot jaw 1004 is mounted to the elongate shaft using a pin 1003 which is pressed into a cavity in the elongate shaft 1002, and which is further pressed into a cavity in the pivot jaw 1004.

The tissue dilating tool is used with other tools, in various embodiments. For example, the tools illustrated in FIGS. 9-10 are useful for implanting an implantable device, and opening a passage into a patient, in various embodiments. One example includes inserting a delivery tool attached to a stylet at least partially into a patient. In varying methods, the stylet is removed and the delivery tool remains at least partially disposed in the patient and the tissue expanding tool is inserted in the delivery tool. The inserted dilation tool is manipulated to vary the jaws using the graspable handle. This procedure expands tissue in the patient for further insertion, dilation, and ultimately implantation of the implantable device.

Continuing with the example, the present subject matter includes guiding an implantable device into the delivery tool, the implantable device having an expandable element. The example includes inserting the expandable element with the delivery tool, and in some cases beyond the delivery tool, and into the tissue of the patient. A physician can position the implantable device in a desired position by positioning the implantable device using a push-wire. In one method, the implantable device is placed in section 806 of the delivery tool, and a push wire is inserted into the delivery device and received by the implantable device. The implantable device can be positioned by a number of techniques, including, but not limited to, visual positioning, ultrasound, palpitation and fluoroscopy. One or more elements and/or implantable devices can be implanted. The push-wire and the delivery tool are removed, the septum or septa are positioned subcutaneously, and any incisions are sutured. Other positioning methods are possible without departing from the scope of the present subject matter.

After implantation the one or more expandable elements can be inflated to at least partially coapt the body lumen. In one embodiment, a syringe and needle are used to introduce flowable material into or withdraw flowable material from the implantable device. The device can be adjusted to increase or decrease the inflation of the one or more expandable elements as long as it is in the patient.

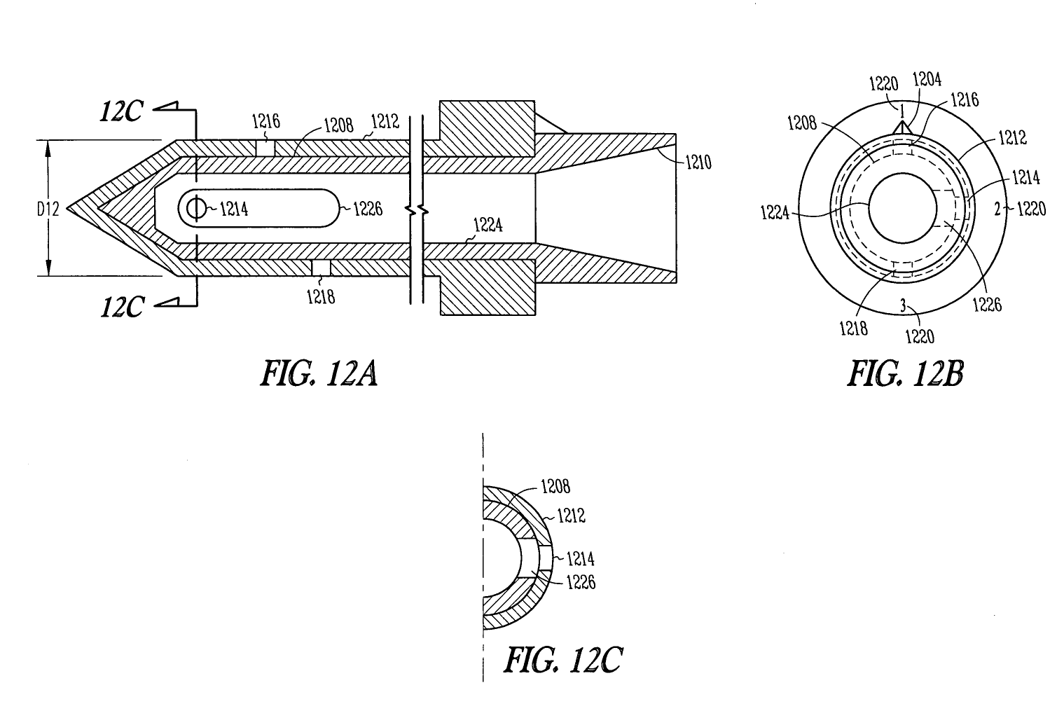

FIGS. 12A-C illustrate a device for adjusting an expandable element, according to one embodiment of the present subject matter. In various embodiments, the device is shaped like a standard hypodermic needle. Various examples include a diameter D12 ranging from about 20 gauge in diameter to about 25 gauge in diameter. Other devices capable of introducing and withdrawing flowable material are possible and other dimensions may be used without departing from the scope of the present subject matter.

FIG. 12A illustrates a front view cross section of an apparatus for adjusting a continence device, according to one embodiment of the present subject matter. Various examples include a first hypodermic needle 1212 shaped like an open-ended cylinder having an interior volume. In various embodiments, the first hypodermic needle 1212 includes one or more apertures 1216 disposed along the length of the first hypodermic needle 1212.

Some embodiments include one or more apertures 1216 disposed at incremental distances along the length of the first open-ended cylinder. One example includes a first aperture 1214 located a first distance from the tip of the first hypodermic needle, a second aperture 1216 located a second distance from the tip of the first hypodermic needle, and a third aperture 1218 located a third distance from the tip of the first hypodermic needle, with the second distance being greater than the first distance, and the third distance being greater than the second distance.

Various embodiments include a second hypodermic needle 1208 shaped like an open-ended cylinder and rotatably mated to the interior volume of the first hypodermic needle 1212, the second hypodermic needle having an elongate opening 1226 disposed along its length. In varying embodiments, the elongate opening is a slot. The slot, in various embodiments, is sized to mate with an aperture in the first hypodermic needle 1212. In varying embodiments, the second hypodermic needle 1208 rotates inside the first hypodermic needle 1212 such that the elongate opening 1226 can align with a single aperture in the first hypodermic needle.

The device, in various embodiments, includes a lumen 1224 defined by the second hypodermic needle, and terminating in a connector 1210. In varying embodiments, the connector 1210 is a Leur connector, but other types of connections are within the scope of the present subject matter. By sealably connecting to the connector 1210, a user can control flowable material between the connector 1210 and the lumen 1224 through the elongate opening 1226, and through a selected aperture, such as aperture 1214, 1216, or 1218. In this manner, the flowable material may be selectively directed through one of multiple apertures in a syringe process.

FIG. 12B illustrates a side view cross section of an apparatus for adjusting a multiport implantable device, according to one embodiment of the present subject matter. The example includes various apertures 1214, 1216, 1218 spaced around the first hypodermic needle 1212. In the example pictured, alignment with aperture 1214 is indicated on the device by an first indicator 1220 disposed on the first hypodermic needle 1212, which is in alignment with a second indicator 1204, disposed on the second hypodermic needle 1208.

FIG. 12C illustrates a side view cross section of an apparatus for adjusting a continence device, the cross section taken at line 12C in FIG. 12A. The view illustrates an alignment between a first hypodermic needle 1212, and a second hypodermic needle 1208, where elongate opening 1226 is aligned with aperture 1214. The second hypodermic needle 1208 is rotatably disposed in the first hypodermic needle 1212, allowing elongate opening 1226 to align with a variety of apertures at different radial and linear positions on the first hypodermic needle 1212.

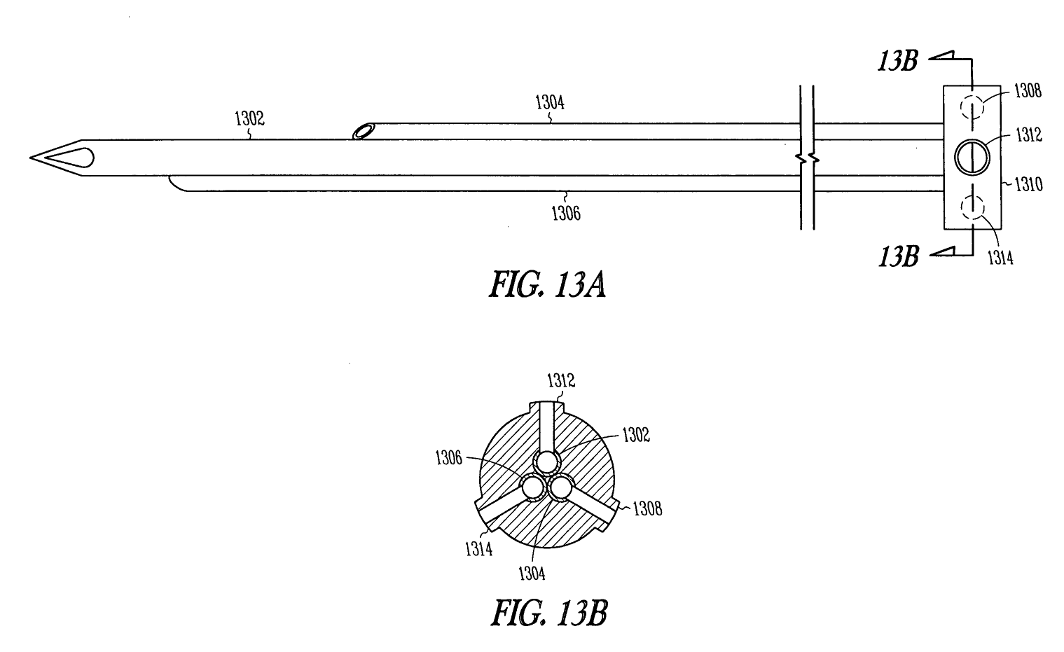

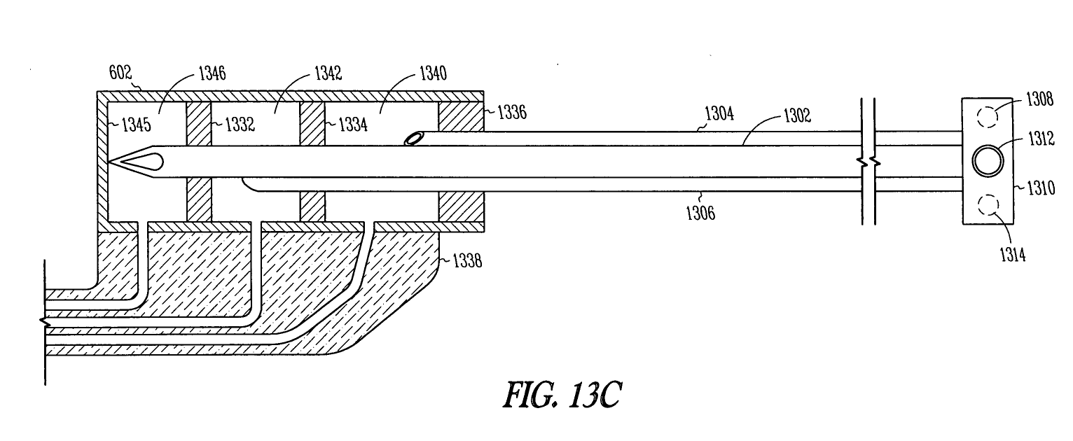

FIGS. 13A-C illustrate a device for adjusting an expandable element, according to one embodiment of the present subject matter. FIG. 13A illustrates, in various embodiments, an apparatus including a manifold 1310 with a plurality of passageways extending between a plurality of first ports 1312, 1314, 1308, and a plurality of hypodermic needles 1302, 1304, 1306. Although three first ports and three hypodermic needles are pictured in the illustration, other embodiments may include different numbers of first ports and hypodermic needles without departing from the scope of the present subject matter.

In some embodiments, multiple hypodermic needles extend away from the manifold 1310 in parallel, terminating at different linear distances. For example, one embodiment includes a first hypodermic needle 1302, which is longer than a second hypodermic needle 1306, the second hypodermic needle 1306 being longer than a third hypodermic needle 1304. Varying designs can deliver fluids simultaneously or independently to one or more individual locations along the length of the device.

FIG. 13B illustrates a cross section view 13B taken from FIG. 13A. In various embodiments, passages extend from the first ports 1308, 1312, 1314 to the hypodermic needles 1304, 1302, 1306, respectively. In various embodiments, the passages are shaped for one or more Leur connections. Other connections are possible in various embodiments without departing from the scope of the present subject matter.

FIG. 13C illustrates a device for adjusting an implantable device in a use position, according to one embodiment of the present subject matter. In the example shown, the adjustment device is coupled with a fill-port block 602. The fill-port block includes a first self-sealing septum 1336 defining a first chamber 1340, a second self-sealing septum defining a second chamber 1342, and a third self-sealing septum 1332 defining a third chamber 1346. In varying embodiments, the first, second, and third chambers extend to passageways in an elongate conduit 1338. While inserted into the fill-port block, the apparatus is useful to communicating fluid to the first, second, and third chambers simultaneously, or individually, in various embodiments. The conduit 1338 is connected to first, second, and third expandable elements, respectively (not shown in FIG. 13C). It is understood that different implantable devices can include one or more expandable elements in various embodiments, and the present subject matter is not limited to three-element devices.

In various embodiments, the design of the fill-port block allows a user to insert the adjustment apparatus without visual feedback. For example, a user can insert the adjustment apparatus until forward motion is substantially restricted, with the needle 1302 contacting a back wall 1345. Additional aid in manipulation can be realized using fluoroscopic tints applied to various aspects of any device to guide manipulation during fluoroscopy.

FIG. 14A illustrates a front view of a nonadjustable element for implantation in a patient, according to one embodiment of the present subject matter. In various embodiments, the nonadjustable element is adapted for implantation exclusively, or in combination therapy with other aspects of therapy disclosed in the present application, and in related applications. One or more nonadjustable elements are useful in therapeutic embodiments where post-operative adjustment is not needed. For example, in one embodiment, the nonadjustable element is implanted one side of a body lumen, while an expandable element is implanted contralateral the lumen. Various benefits stem from procedures using nonadjustable elements, including improved coaptation.

In various embodiments, the nonadjustable element is stainless steel. In an additional embodiment, the nonadjustable element is constructed from an acetal resin, such as polyacetyl. Additional materials are within the scope of the present subject matter, including materials comprised of and/or coated with an antimicrobial material. In various embodiments, placement in the patient is aided through use of a tool such as the tool illustrated in FIG. 9. In one embodiment, the nonadjustable element is compressed for passage through a tool such as that illustrated in FIG. 9, expanding in a use position. Additionally, placement is aided through the use of a tool such as the tool illustrated in FIG. 11. For example, tissue is expanded creating an anatomical pocket adapted to mate with a nonadjustable element. These uses are provided for illustration and are not exhaustive or exclusive.

The nonadjustable element, in one embodiment, is cylindrical, with a middle section 1402, a first end section 1404, and a second end section 1406. In varying embodiments, the sections including varying diameters, and the transitions between them including linear shapes and curved shapes. In one example, a large middle section 1402 is placed near a body lumen, while a first section 1404 and a second section 1406 are used for positioning.

FIG. 14B illustrates a side view of an element for implantation in a patient, according to one embodiment of the present subject matter.

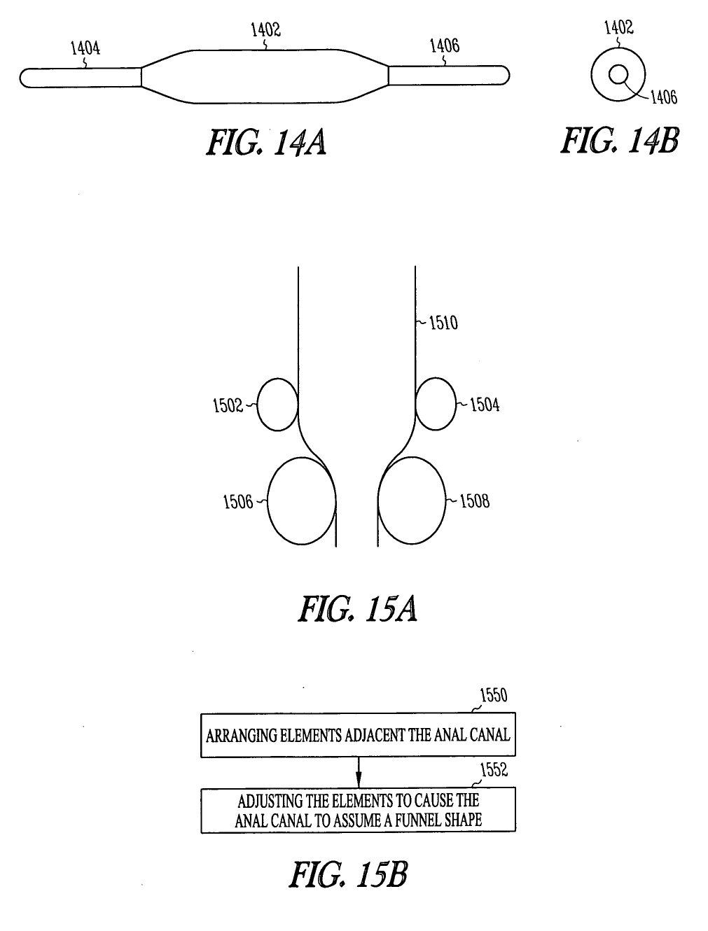

FIG. 15A illustrates an implanted device near a funnel shaped anal canal, according to one embodiment and application of the present subject matter. In various embodiments, the implanted device includes a first expandable element 1502, a second expandable element 1506, a third expandable element 1504, and a fourth expandable element 1508. One embodiment positions the four expandable elements near the anal canal 1510 to encourage a funnel shape. In one embodiment, the four expandable elements are arranged in a coplanar, quadrilateral configuration, with two expandable elements on one side of the anal canal 1510, and two expandable elements contralateral the anal canal 1510. By positioning the elements as such, and through adjustment, including, but not limited to, post-operative adjustment using fluoroscopy, a physician may obtain a funnel shaped anal canal 1510, which restores natural function of the anal canal 1510 and continence.

FIG. 15B illustrates one method for fecal continence, according to one embodiment of the present subject matter. Embodiments include arranging elements adjacent the anal canal 1550. Various embodiments include arranging a plurality of expandable elements quadrilaterally and coplanar with at least two expandable elements positioned near one side of the anal canal, and with at least two expandable elements positioned near the contralateral side of the anal canal. Additional embodiments include adjusting the expandable elements to induce the anal canal to assume a funnel shape 1552. While the elements shown are coplanar, it should be understood that the elements can also be arranged in a non-coplanar configuration to induce a funnel shape.

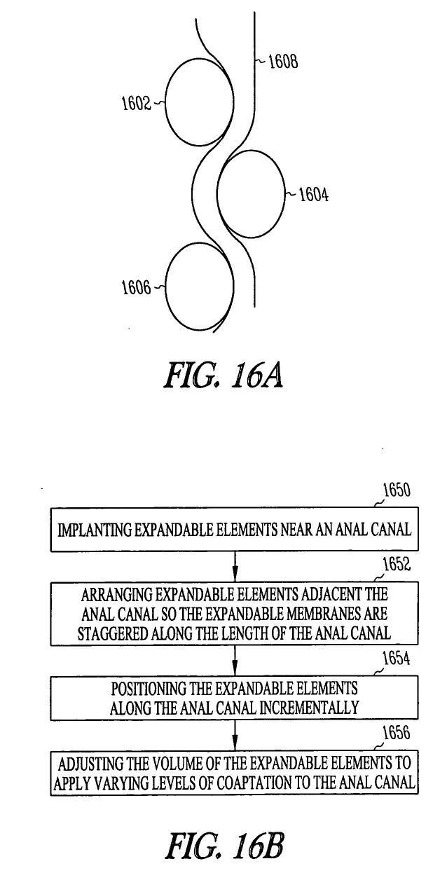

FIG. 16A illustrates an implanted device and a coapted anal canal, according to one embodiment and application of the present subject matter. As demonstrated, the implanted device includes a first expandable element 1602, a second expandable element 1604, and a third expandable element 1606 positioned along the anal canal 1608 in an alternating pattern, resulting in an anal canal 1608 shaped like a sinusoidal curve. In one embodiment, the three elements are located approximately in the same plane, but other orientations are within the scope of the present subject matter. Overall, by implanting expandable elements along the anal canal 1608, and by adjusting the implanted elements, a physician can adjust the function of the anal canal 1608 to restore continence and perhaps suit patient preference.

FIG. 16B illustrates one method for fecal continence, according to one embodiment of the present subject matter. In various embodiments, the present subject matter includes implanting a plurality of individual expandable elements near an anal canal 1650. Various examples include arranging expandable elements along the anal canal so the expandable elements are staggered along the length of the anal canal 1652. For example, various embodiments include arranging the plurality of expandable elements so they are bisected by a unitary reference plane which bisects the anal canal. The plurality of expandable elements are positioned along the anal canal incrementally 1654. Volume of the plurality of expandable elements is adjusted to apply varying levels of coaptation to the anal canal 1656, thereby restoring natural function and continence.

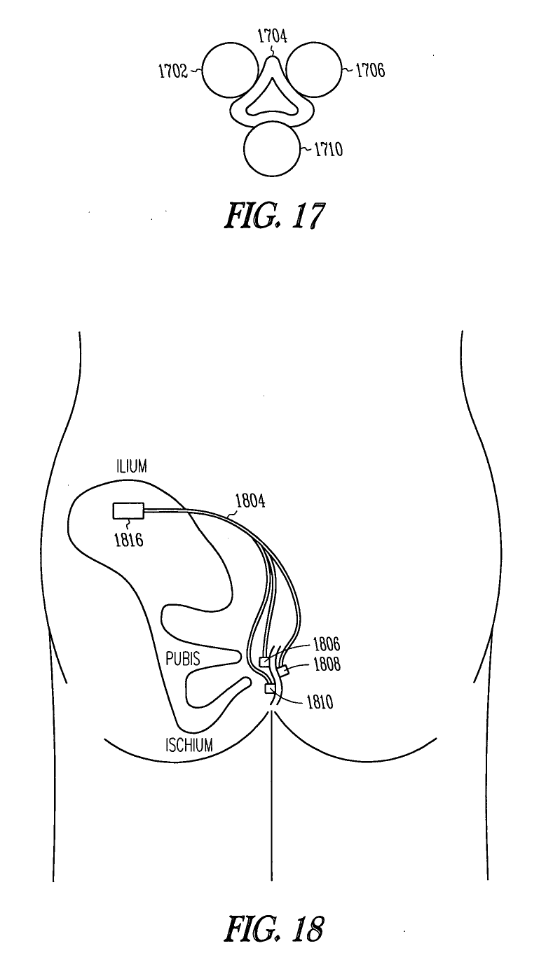

FIG. 17 illustrates a cross section of an implanted device and a coapted anal canal, according to one embodiment of the present subject matter. In varying embodiments, an implantable device including a first expandable element 1702, a second expandable element 1706, and a third expandable element 1710 is implanted near a body lumen 1704. In varying embodiments, the expandable elements are disposed at varying positions around the body lumen 1704. In one embodiment, the expandable elements are coplanar, and arranged around the body lumen at approximately 120 degree increments.

FIG. 18 illustrates a schematic of an implanted fecal continence device, according to one embodiment and application of the present subject matter. In one example implantation, a fill-port block 1816 is implanted in the patient near the patient's hip. An elongate conduit 1804 extends from the fill-port block 1816 to one or more expandable elements. The illustrated schematic includes a first expandable element 1806, a second expandable element 1808, and a third expandable element 1810. Various configurations of the one or more expandable elements are within the scope of the present subject matter. Subcutaneous placement of the fill-port block 1816 enables post-operative adjustment of the one or more expandable elements by using an adjusting device such as a hypodermic needle inserted through the skin near the hip. Other apparatus and placements may vary without departing from the scope of the present subject matter.

Varying embodiments of the present subject matter include expandable elements implanted near the anal canal, with one or more elongate expandable elements oriented approximately parallel the anal canal. For example, in one embodiment, an elongate conduit extends from a fill-port to one or more expandable elements. In various embodiments, the one or more expandable elements are elongate, with a near portion and a far portion, the near portion oriented toward the rectum, the far portion extending toward the anal sphincter, and the balance extending along the anal canal. In some embodiments, the element is positioned near fibrous membranes comprising the anal canal and the anal sphincter. By orienting the one or more expandable elements so they extend along the anal canal, a desired level of anal canal coaptation can be achieved and continence is restored.

FIG. 19 illustrates a method for using anal manometry in implantation of a fecal continence device, according to one embodiment of the present subject matter. In varying embodiments, surgical methods used to implant the devices of the present subject matter benefit from manometry. In varying embodiments, by using anal manometry, a user may measure the pressure changes occurring from adjustment of one or more expandable elements. The measurement includes a baseline measurement of an anal canal which is not influenced by an inflated expandable element 1950. Volume of the expandable elements is adjusted 1952, in various embodiments. Additional measurements are performed to record the influence of adjustment 1954, in various embodiments. By monitoring the pressure, an increased level of control during adjustment is possible, which can result in a reduced number of adjustments to achieve a desired level of continence of a body lumen such as the anal canal.

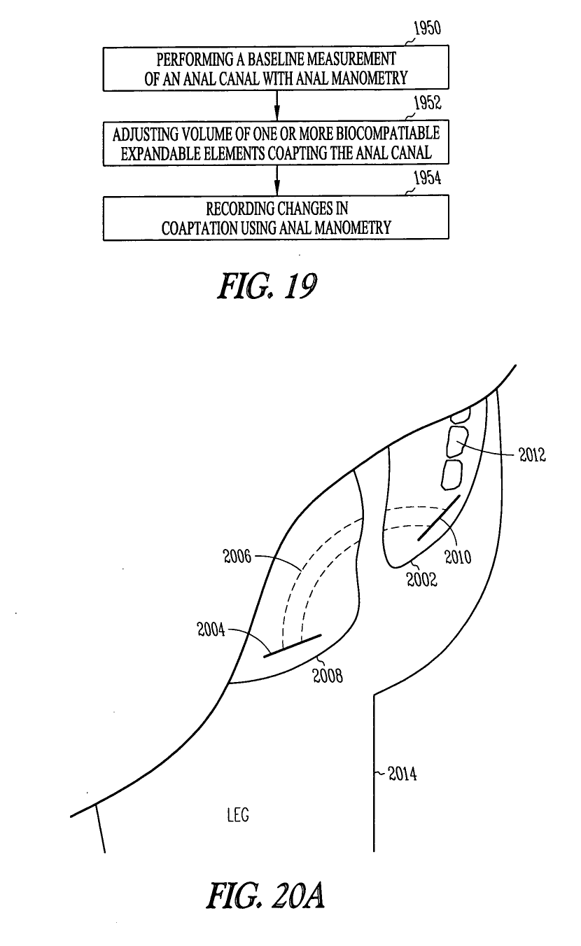

FIG. 20A illustrates a cross section of a subject's anatomy. A patient anal sphincter 2002, a perineum 2008, a spine 2012, and a patient leg 2014 are illustrated. Additionally, a passage 2006, illustrated with hidden lines, extends between a perineal incision 2004 and a caudal incision 2010. FIG. 20B illustrates a method for using a caudal incision for implantation of a fecal continence device, according to one embodiment of the present subject matter. In varying embodiments, surgical methods of the present subject matter include implanting one or more devices through a first incision in the perineum 2050. Various embodiments also include creating a caudal incision 2052. A caudal incision, in various embodiments, is an incision positioned between the anus and the sacral region of backbone. Additional embodiments include creating a passage between the perineal incision and the caudal incision 2054. In varying embodiments, a passageway extending from the first incision to the second incision is used to thread one or more expandable elements into position around an anal canal 2056.

Various apparatus and method described here include multiple benefits. For example, because the disclosed apparatus do not include a cuff-shaped design, they reduce chances of obstruction. Additionally, the present apparatus and method provide less interference with normal blood perfusion in the areas near the implant than other designs. Further benefits include a reduced likelihood of tissue erosion, infection, or tissue atrophy. These reduced symptoms result in lower chances of related weakening of tissues which naturally support body lumens. The post-operative adjustability of the apparatus discussed here additionally decreases the risk of infection, as fewer surgeries are needed. Also, the passive nature of the apparatus, and the manner, shape, and configurations in therapeutic use, benefit patients by increasing natural feeling during voiding. The present method and apparatus assists a patient in selecting when to void and restores natural lumen function. Overall, a patient benefiting from the present subject matter can control continence as they would naturally, prior to their suffering anatomical dysfunction.

Although specific embodiments have been illustrated and described herein, it will be appreciated that the present subject matter includes apparatus and method that vary from the examples provided herein without departing from the scope of the present disclosure. This application is intended to cover adaptations or variations of the present subject matter. It is to be understood that the above description is intended to be illustrative, and not restrictive. The scope of the present subject matter should be determined with reference to the appended claims, along with the full scope of equivalents to which such claims are entitled.

What is claimed is:

1. An apparatus, comprising:
an expandable element;
a fill port block including a self-sealing septum; and
a conduit providing fluid communication between the fill port block and the expandable element,
wherein the expandable element is adapted to become toroidal in shape when at least partially filled and wherein the apparatus is adapted for implantation.

2. The apparatus of claim 1, wherein the expandable element is adapted to expand to a predetermined shape.

3. The apparatus of claim 1, wherein the expandable element includes silicone elastomer.

4. The apparatus of claim 1, wherein the expandable element includes polyurethane elastomer.

5. The apparatus of claim 1, wherein the fill port block includes a needle stop.

6. The apparatus of claim 1, further comprising a second expandable element and a second conduit sealingly connected to the apparatus and providing fluid communication to the second expandable element.

7. The apparatus of claim 6, wherein the second conduit is connected to the fill port block to provide fluid communication to the second expandable element.

8. The apparatus of claim 6, wherein the second conduit is connected to the expandable element to provide fluid communication to the second expandable element.

9. The apparatus of claim 6, wherein the expandable element is a different shape than the second expandable element.

10. The apparatus of claim 6, wherein the fill port has a single self-sealing septum.

11. The apparatus of claim 6, wherein the fill port has a plurality of self-sealing septums.

12. The apparatus of claim 6, wherein the second expandable element is cylindrical or curved, or asymmetrical.

13. The apparatus of claim 6, further comprising a third expandable element and a third conduit sealingly connected to the apparatus and providing fluid communication to the third expandable element.

14. The apparatus of claim 13, wherein the fill port has a single self-sealing septum.

15. The apparatus of claim 13, wherein the fill port has a plurality of self-sealing septums.

16. The apparatus of claim 1, wherein the conduit is between 5 and 20 centimeters.

17. An apparatus, comprising:
an expandable element;
a fill port block including a self-sealing septum; and
a conduit providing fluid communication between the fill port block and the expandable element,
wherein the expandable element is adapted to become curved in shape when at least partially filled and wherein the apparatus is adapted for implantation.

18. The apparatus of claim 17, wherein the expandable element is adapted to expand to a predetermined shape.

19. The apparatus of claim 17, wherein the expandable element includes silicone elastomer.

20. The apparatus of claim 17, wherein the expandable element includes polyurethane elastomer.

21. The apparatus of claim 17, wherein the fill port block includes a needle stop.

22. The apparatus of claim 17, further comprising a second expandable element and a second conduit sealingly connected to the apparatus and providing fluid communication to the second expandable element.

23. The apparatus of claim 22, wherein the second conduit is connected to the fill port block to provide fluid communication to the second expandable element.

24. The apparatus of claim 22, wherein the second conduit is connected to the expandable element to provide fluid communication to the second expandable element.

25. The apparatus of claim 22, wherein the expandable element is a different shape than the second expandable element.

26. The apparatus of claim 22, wherein the fill port has a single self-sealing septum.

27. The apparatus of claim 22, wherein the fill port has a plurality of self-sealing septums.

28. The apparatus of claim 22, wherein the second expandable element is cylindrical or toroidal, or asymmetrical.

29. The apparatus of claim 22, further comprising a third expandable element and a third conduit sealingly connected to the apparatus and providing fluid communication to the third expandable element.

30. The apparatus of claim 29, wherein the fill port has a single self-sealing septum.

31. The apparatus of claim 29, wherein the fill port has a plurality of self-sealing septums.

32. The apparatus of claim 17, wherein the conduit is between 5 and 20 centimeters.

33. An apparatus for filling a plurality of septums of a fill port, comprising:
a hypodermic needle body; and
needle means for communicating fluid with the plurality of septums of the fill port.

34. The apparatus of claim 33, wherein the needle means includes one or more apertures disposed length of the needle means.

35. The apparatus of claim 33, wherein the needle means includes apertures along the length of the device.

36. A method, comprising:
connecting a plurality of adjustable elements to a plurality of conduits; and
connecting at least one conduit of the plurality of conduits to a self-sealing septum to form a closed system which is adapted for adjusting volume of the adjustable elements based on fluids exchanged between the system and an external source.

37. The method of claim 36, further comprising connecting the external source to the septum.
